(19) United States
(12) Patent Application Publication
(10) Pub. No.: US 2014/0121554 A1
(43) Pub. Date: May 1, 2014

(54) SEIZURE DETECTION DEVICE AND SYSTEMS

(71) Applicant: The Johns Hopkins University, Baltimore, MD (US)

(72) Inventors: Sridevi V. Sarma, McLean, VA (US); Sabato Santaniello, Baltimore, MD (US); Samuel P. Burns, Philadelphia, PA (US); Munther Dahleh, Cambridge, MA (US)

(73) Assignee: The Johns Hopkins University, Baltimore, MD (US)

(21) Appl. No.: 14/065,714

(22) Filed: Oct. 29, 2013

Related U.S. Application Data
(60) Provisional application No. 61/719,838, filed on Oct. 29, 2012.

(51) Int. Cl. A61B 5/04

(52) U.S. Cl. CPC A61B 5/04012
USPC 600/544

(57) ABSTRACT

A neurostimulation device includes a plurality of electrodes adapted to be electrically connected to a subject to receive multichannel electrical signals from the subject's brain, a multichannel seizure detection unit electrically connected to the plurality of electrical leads to receive the multichannel electrical signals, and a neurostimulation unit in communication with the multichannel seizure detection unit. The plurality of electrodes are at least three electrodes such that the multichannel electrical signals are at least three channels of electrical signals, and the multichannel seizure detection unit detects a presence of a seizure based on multichannel statistics from the multichannel electrical signals including higher order combinations than two-channel combinations. Another embodiment of the invention includes determining a singular vector centrality (SVC) for each of the electrodes in order to detect the seizure onset.

### CROSS-REFERENCE TO RELATED APPLICATION

This application claims priority to U.S. Provisional Application 61/719,838 filed on Oct. 29, 2012, the entire contents of which are hereby incorporated by reference.

### GOVERNMENT SUPPORT

This invention was made with government support under ECCS-1346888 awarded by the National Science Foundation. The government has certain rights in the invention.

### BACKGROUND

1. Field of Invention

The field of the currently claimed embodiments of this invention relates to seizure detection devices and systems.

2. Discussion of Related Art

Epilepsy has a prevalence of about 1% in children and adults, and is characterized by chronically recurring seizures without clear precipitants. A seizure is a finite-time episode of disturbed cerebral function with abnormal, excessive, and synchronous electrical discharges in large groups of cortical neurons. Disturbances may be associated with debilitating phenomena (e.g., convulsions, low responsiveness, etc.) or remain clinically unapparent, have a duration ranging from seconds to minutes, and may be followed by post-ictal periods of confusion, psychosis, or sensory impairment, which can last up to several hours. Epilepsy in children is associated with problems including academic achievement, behavioral and emotional adjustment, and social competence, and contributes to 0.5% of the global economic burden of diseases.

Despite a large variety of medications available to treat epilepsy, 25% of children (30% of adults) are drug-resistant. Furthermore, since medications are administered without any knowledge of an impending seizure, overtreatment is frequent and may lead to increased morbidity, psychosocial handicaps and mortality. Children are the most at risk for developing long-term morbidity, as poorly controlled seizures can affect long-term cognitive development and function. Alternative treatments for drug-resistant patients include surgical resection of the epileptogenic zone and neurostimulation. Surgical resection is widely accepted but is not always possible and its success mostly depends on the correct localization of the epileptic focus and the specific cortical area to be resected. Chronic open- and closed-loop neurostimulation are still under clinical trials for adults, and although the results are encouraging, their therapeutic effectiveness critically depends on electrode placement, coverage, morphology of seizure, and most importantly seizure onset detection.

The accurate detection of seizure onsets from sequential iEEG (intracranial electroencephalography) measurements is fundamental for the development of both responsive neurostimulation and effective patient-warning devices. Several OSD (online seizure detection) algorithms have been proposed thus far and though they are highly sensitive (large number of true positives), these algorithms generally have low specificity (large number of false positives), which limits their clinical use. NeuroPace Inc. has pioneered the development and testing of a closed-loop device, the RNS™ system, which automatically detects an approaching seizure by monitoring two iEEG channels and responds with high frequency periodic stimulation in drug-resistant epilepsy patient. Despite promising results in small populations of patients after short-term follow ups (less than 2 years), a recent long-term (5 years) study has indicated that the device reduces the number of seizures by 50% in less than 30% of the patients (reduction computed vs. the baseline pre-treatment condition), which is about as effective as a new medication in patients with drug-resistant partial seizures. Although the detection algorithms can be tuned for seizures in a given patient, these simple algorithms lack specificity with many detections of inter-ictal activity that are not destined to evolve into electrical or clinical seizure activity. Since detections result in activation of closed-loop therapy, stimulations are frequently delivered when no seizure occurs. While no significant side effects of stimulation were observed in the RNS™ trial, increased stimulation frequency can dramatically reduce battery life (typically to 1-2 years). In other studies, there are reports of possible consequences of repetitive stimulation including depression, memory impairment, and confusion.

The lack of specificity of current OSD algorithms including the one implemented in the RNS™ system presumably occurs because (i) they compute statistics from 1-2 channels at a time that may not capture network dynamics of the brain, and/or (ii) the detection thresholds are not optimized to maximize OSD performance. An optimal detection of the seizure onset, instead, would have higher specificity and ultimately improve the performance of closed-loop therapies.

Automatic online seizure detection (AOSD) in intractable epilepsy has generated great interest in the last thirty years and is a fundamental step toward the development of anti-epileptic responsive neurostimulation. Pioneering works in the late 1970s and 1980s showed that seizures can be automatically separated from inter-ictal activity, and since then, several approaches to AOSD have been proposed by exploiting either scalp or intracranial EEG recordings, single or multi-channel analysis, linear or nonlinear features.

One study used a wavelet-based decomposition of selected iEEG recordings to (i) separate the seizure-related component from the background noise, (ii) track the ratio between these components in the time-frequency domain, and (iii) detect a seizure when such a ratio crosses a fixed threshold for a sufficiently long time. Parameters of the detection method (e.g., threshold, duration of the supra-threshold condition, etc.) can be either fixed or adaptive. Fixed threshold-based approaches were also proposed, where the threshold was applied to linear spectral features of the iEEG recordings.

Another study proposed a probabilistic framework for seizure detection using scalp EEG and iEEG recordings. For each channel, amplitude and energy measures in multiple frequency bands are computed via wavelet decomposition and the corresponding probability distribution function is estimated. Then, the probability of a seizure is conditioned on the value of such measures and estimated via Bayes' rule. A patient-specific threshold is finally applied on this conditional probability of seizure to decide, for each channel, whether a seizure is likely, and a seizure is detected when that threshold is passed in a sufficient number of channels.

More recently, this paradigm has been implemented using sophisticated classification tools. In particular, iEEG channels have been processed individually to extract multiple univariate or bivariate features in the time, frequency, or wavelet domain. Then, for each channel, the features have been combined into vectors and classified via support vector machines (SVMs), principal components analysis (PCA), artificial neural networks (ANNs), fuzzy logics, or pattern recognition tools. Finally, decisions made for different channels are combined or ranked to ultimately determine whether a seizure has occurred. As a variation to this paradigm, it has also been proposed to merge the features extracted from different channels into one single vector and then to apply a classification rule on this vector.

Overall, the paradigm described above solves the AOSD problem by computing a statistic from a few iEEG measurements at a time, and then constructing a threshold or a classification rule that, based on this statistic, determines whether or not a seizure has occurred (FIG. 2A). The choice of the threshold is traditionally supervised and depends on the fluctuations of the statistic, the specific patient, or the electrode position, and requires long training sessions to be more accurate. Sophisticated classifiers use optimization tools to generate unsupervised criteria that separate the feature space into dominant ictal and non-ictal regions, but they do not include penalties that account for specific performance goals (e.g. minimize false positives). As a result, all these thresholds trigger too many false alarms when applied to test data. Consequently, all efforts put towards improving AOSD algorithms have been in either identifying better statistics with fancy signal processing and/or in implementing more sophisticated classifiers borrowed from the machine learning community. The fundamental problem with this paradigm is that detection performance is not measurable until after implementation (“algorithm defines performance”). There thus remains a need for improved seizure detection devices and systems.

### SUMMARY

A multichannel seizure detection system in accordance with an aspect of the present invention includes a signal interface adapted to receive multichannel electrical signals from said subject's brain. The system also includes a data processor configured to receive said multichannel electrical signals and detect the onset of a seizure. Additionally, the onset of a seizure is determined using the singular vector centrality (SVC) of each one of the channels comprising the multichannel electrical signals.

In accordance with another aspect of the present invention, a method for detecting seizures in a subject includes obtaining multichannel electrical signals from said subject's brain. The method includes transmitting the multichannel electrical signals from said subject's brain to a computing device, wherein said computing device is loaded with a non-transitory computer readable medium. The non-transitory computer readable medium is programmed for detecting the presence of an epileptogenic zone (the zone where a seizure starts) and determining the seizure onset by using the singular vector centrality (SVC) of each one of the channels comprising the multichannel electrical signals.

### BRIEF DESCRIPTION OF THE DRAWINGS

Further objectives and advantages will become apparent from a consideration of the description, drawings, and examples.

FIG. 1 is a schematic illustration of a neurostimulation device according to an embodiment of the current invention.

FIGS. 2A and 2B contrast conventional approaches (top) with an embodiment of the current invention (bottom: Multivariate QD [Quickest Detection] approach to AOSD).

FIGS. 3A and 3B illustrate some concepts of the current invention. (A) three-node network with loose connections under 2 measures (2 edges between each pair of nodes) and the corresponding non-square connectivity matrix A, singular values, σ(A), and leading singular vector u₁(A). (B) Nodes 1 and 2 of the network are strongly connected under both measures (thick edges). The first two singular values of the connectivity matrix B are much larger than the third one and the first two components of first singular vector u₁(B) are significantly larger in magnitude than the third component.

FIGS. 4A-4D provide the first singular values σ₁ and the correspondent first singular vectors u₁ around an ictal period (a seizure, gray background) in patient 1 (seizure s₁, A), 2 (s₁, B), 3 (s₃, C), and 4 (s₃, D) of 4 patients, who represent a preliminary dataset. FIG. 4E shows u₁ vs. time for patient 4. FIG. 4F provides average u₁ of the first seizure projected onto subsequent seizures.

FIG. 5A is a schematic illustration of a m=2 state HMM according to an embodiment of the current invention. FIG. 5B shows the sampling probability distribution function of the observations zₖ collected in state xₖ=0 (non-ictal) and xₖ=1 (ictal). Data collected from Patient 2 in the preliminary dataset. FIG. 5C shows AOSD formulated as a feedback control problem according to an embodiment of the current invention.

FIG. 6 shows history-dependent values of qₒ(zₖ|Hₖ) and q₁(zₖ|Hₖ) at the onset of an annotated seizure (gray background).

FIGS. 7A and 7B provide QD on validation data according to an embodiment of the current invention. The electrographic onset (red line), the correspondent QD estimation (circles), and threshold Fₖ (green and blue lines for different values of parameter γ which trades off specificity/delay) for Patient 2 (A) and 4 (B). Plots for seizures s₃ in both patients. In bottom plots, horizontal dashed black line is threshold for the BE (Bayesian Estimator) detector.

FIGS. 8A-8C illustrate graphical views related to determining a singular vector centrality (SVC) for nodes of a testing device to determine the seizure onset in the epileptogenic zone, according to an embodiment of the present invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

The following defines various abbreviations and terms used throughout the description:
iEEG: intracranial EEG is an invasive technique that records electroencephalography (EEG) signals directly from the human cortex, as opposed to surface recordings in scalp-EEG. This is achieved either by using subdural grids or strips of electrodes placed directly on the surface of the cortex (also known as Electrocorticography), or using multi-lead depth electrodes.
ECoG: electrocorticography (see iEEG).
OSD: online seizure detection. An algorithm that measures iEEG or scalp-EEG activity sequentially and estimates the onset times of each seizure.
EMU: epilepsy monitoring unit.
Ictal: ictal refers to a physiologic state or event during a seizure episode.
QD: quickest detection is a change-point detection algorithm that minimizes detection delay and probability of false alarm.
SVD: in linear algebra, the singular value decomposition (SVD) is a factorization of a real or complex matrix, with many useful applications in signal processing and statistics.
HMM: hidden Markov model. A specific model where the states are unobservable but the outputs in each state are observable/measureable. It is a statistical Markov model in which the system being modeled is assumed to be a Markov process with unobserved (hidden) states. An HMM can be considered as the simplest dynamic Bayesian network. In a regular Markov model, the state is directly visible to the observer, and therefore the state transition probabilities are the only parameters. In a hidden Markov model, the state is not directly visible, but output, which is dependent on the state, is visible. Each state has a probability distribution over the possible output tokens. Therefore the sequence of tokens generated by an HMM gives some information about the sequence of states. Note that the adjective “hidden” refers to the state sequence through which the model passes, not to the parameters of the model; even if the model parameters are known exactly, the model is still “hidden”.
SVM: support vector machine. A support vector machine (SVM) is a concept in computer science for a set of related supervised learning methods that analyze data and recognize patterns, used for classification and regression analysis. The standard SVM takes a set of input data and predicts, for each given input, which of two possible classes comprises the input, making the SVM a non-probabilistic binary linear classifier. Given a set of training examples, each marked as belonging to one of two categories, an SVM training algorithm builds a model that assigns new examples into one category or the other. An SVM model is a representation of the examples as points in space, mapped so that the examples of the separate categories are divided by a clear gap that is as wide as possible. New examples are then mapped into that same space and predicted to belong to a category based on which side of the gap they fall on.
PCA: principal component analysis. PCA is a mathematical procedure that uses an orthogonal transformation to convert a set of observations of possibly correlated variables into a set of values of uncorrelated variables called principal components. The number of principal components is less than or equal to the number of original variables. This transformation is defined in such a way that the first principal component has as high a variance as possible (that is, accounts for as much of the variability in the data as possible), and each succeeding component in turn has the highest variance possible under the constraint that it be orthogonal to (uncorrelated with) the preceding components.
ANN: artificial neural network, usually called also neural network (NN). An ANN is a mathematical model or computational model that is inspired by the structure and/or the functional aspects of biological neural networks. A NN consists of an interconnected group of artificial neurons, and it processes information using a connectionist approach to computation.
DP: dynamic programing. In mathematics and computer science, dynamic programming is a method for solving complex problems by breaking them down into simpler sub-problems. It is applicable to problems exhibiting the properties of overlapping sub-problems, which are only slightly smaller and optimal sub-structure. When applicable, the method takes far less time than naive methods. The key idea behind dynamic programming is quite simple. In general, to solve a given problem, we need to solve different parts of the problem (sub-problems) and then combine the solutions of the sub-problems to reach an overall solution. Often, many of these sub-problems are really the same. The DP approach seeks to solve each sub-problem only once, thus reducing the number of computations. This is especially useful when the number of repeating sub-problems is exponentially large.

As described in the background section, according to conventional approaches, OSD is solved by (i) computing a statistic from iEEG measurements that captures changes in brain activity at the seizure onset, and (ii) by constructing a threshold or classification rule that, based on this statistic, determines whether or not a seizure has occurred. The choice of the threshold is typically supervised and depends on the fluctuations of the statistic, the specific patient, and/or the electrode position. Such thresholds trigger too many false alarms when applied to test data because the statistic and/or threshold does not separate ictal from non-ictal activity well. Furthermore, true positives may be detected with unacceptably long delays. Consequently, all efforts put towards improving OSD algorithms have been in either identifying better statistics with fancy signal processing and/or in implementing more sophisticated classifiers, which have been borrowed from the machine learning community. A problem with this paradigm is that detection performance (e.g., the total number of false positives) is not measurable until after implementation (that is, the algorithm defines performance).

Instead, some embodiments of the current invention take a fresh new approach to OSD. First, we note that classification rules, no matter how fancy, do not account for temporal dependencies and dynamics that exist in neural data. For example, a statistic measured at some time t depends on what values it took on in the past 100-500 milliseconds (ms). Therefore, one should model this evolution—it is not what the iEEG activity looks like at any given moment, but how it got there. Then, predictions from this model should be used to adapt the threshold rule. For example, if the dynamics appear to be evolving toward an ictal state, then the threshold should drop quickly, thus making it easier to detect a seizure onset. However, the threshold should adapt in an unsupervised and optimal way. Optimality should be defined by maximizing detection performance (that is, the performance defines algorithm!).

To address the raised issues, some embodiments of the current invention provide a novel computational framework for OSD that involves (i) constructing multivariate statistics from all electrodes to distinguish between non-ictal and ictal states; (ii) modeling the evolution of these statistics in each state and the state-transition probabilities; and, (iii) developing an optimal model-based strategy to detect transitions to ictal states from sequential neural measurements. This strategy is formulated as the Bayesian Quickest Detection (QD) of the seizure onset and solved by using optimal control tools, which explicitly minimize both the distance between the detected and unequivocal onset times of the seizures and the probability of false alarms. This is a paradigm shift and (i)-(iii) are described in detail below.

Some embodiments of the current invention may enable more robust detection of seizures for closed-loop intervention. Post-hoc review of patient iEEG records (offline seizure confirmation) can also be made more efficient. Overall, the outcomes can lead to more effective treatments, which could potentially avoid fatal accidents, thereby saving lives, extending life-expectancy, and improving the administration of anti-seizure drugs.

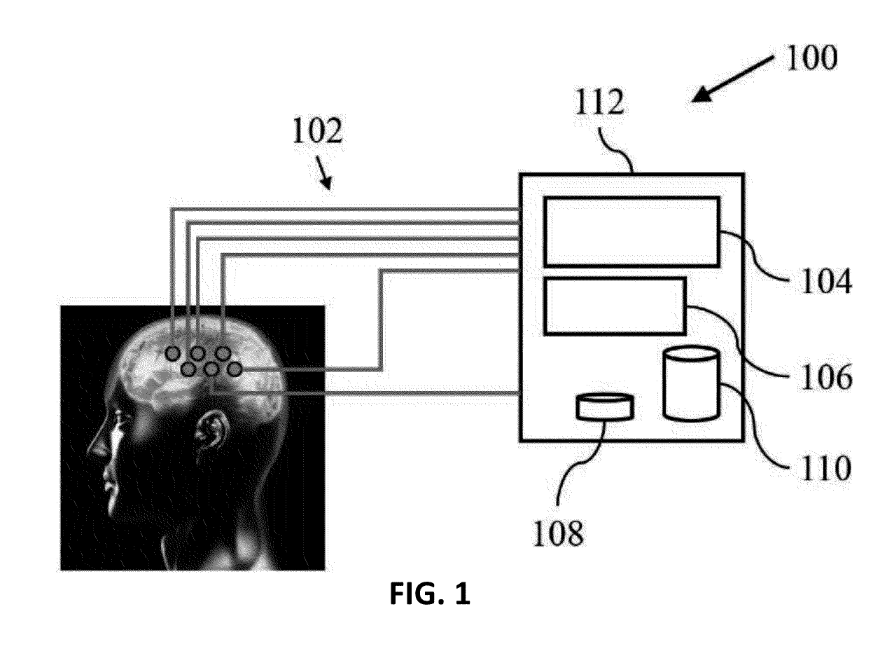

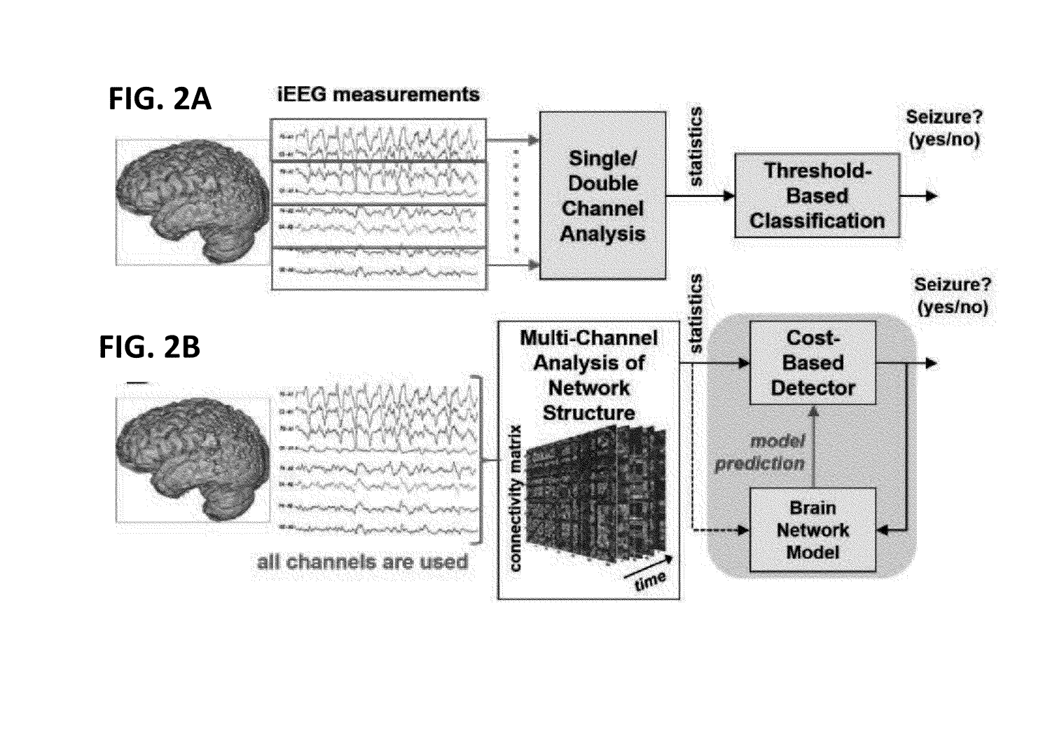

FIG. 1 is a schematic illustration of a neurostimulation device 100 according to some embodiments of the current invention. The neurostimulation device 100 includes a plurality of electrodes 102 adapted to be electrically connected to a subject to receive multichannel electrical signals from the subject's brain, a multichannel seizure detection unit 104 electrically connected to the plurality of electrical leads 102 to receive the multichannel electrical signals, and a neurostimulation unit 106 in communication with the multichannel seizure detection unit 104. The neurostimulation device 100 can also include other components, such as a power supply 108 and/or data memory or storage components 110. The power supply can include a battery or other electrical power storage devices, for example. The multichannel seizure detection unit 104 and/or the neurostimulation unit 106 can be at least partially implemented on a data processor encoded with software, or could be implemented on hard-wired devices, for example. The electronics of the neurostimulation device 100 can be packaged together in a case 112 in some embodiments. The electrodes 102 can be attached externally or intracranially, depending on the application. In some embodiments, the case 112 can be also implantable such that the neurostimulation device 100 is an implantable device. However, the general concepts of the current invention are not limited to only implantable devices. In other embodiments, the neurostimulation device 100 can be an external device with electrodes attached to the subject's scalp.

The plurality of electrodes 102 are at least three electrodes such that the multichannel electrical signals are at least three channels of electrical signals. However, the broad concepts of the current invention are not limited to only three electrodes. In some embodiments, there can be up to 10, 20, 30, 50 or even more electrodes, depending on the particular application.

The multichannel seizure detection unit 104 detects a presence of a seizure based on multichannel statistics from the multichannel electrical signals including higher order combinations than two-channel combinations. In other words, statistics are not determined for only signals within single channels and correlations between pairs of channels. They are also based on triplets of channels, etc.

In some embodiments of the current invention, multichannel seizure detection unit 104 is configured to model the multichannel electrical signals based on a brain network model. In some embodiments, the brain network model models time-dependent variations of the multichannel statistics. In some embodiments, the brain network model is a hidden Markov model. However, the broad concepts of the current invention are not limited to only hidden Markov models. In some embodiments, the multichannel seizure detection unit 104 is configured to detect the presence of the seizure based on a time-dependent threshold. In some embodiments, the multichannel seizure detection unit 104 is configured to detect the presence of the seizure based on optimizing a cost function. The cost function can be dependent on a time delay between an actual seizure and a prediction of the seizure, for example. In some embodiments, the cost function can be further dependent on the probability of a false positive detection. In some embodiments, the neurostimulation unit 106 can be configured to provide an electrical stimulation to the subject's brain. Alternatively, or in addition, the neurostimulation unit 106 can be configured to provide a chemical stimulation. Alternatively, or in addition, the neurostimulation unit 106 can be configured to provide other types of stimulation such as feedback and/or signals. The neurostimulation unit 106 can be responsive to output from the multichannel seizure detection unit 104 in some embodiments. For example, the multichannel seizure detection unit 104 can trigger the neurostimulation unit 106. In some embodiments, the multichannel seizure detection unit 104 can be used alone without a neurostimulation unit 106 and/or in conjunction with other devices.

### Multivariate Statistics

To identify robust multivariate statistics for seizure detection, multichannel iEEG signals can be processed into generalized non-square connectivity matrices that describe the time-varying spectral dependencies between all the channels over multiple frequency bands. The singular value decomposition (SVD) can be used to extract multivariate statistics (e.g., the leading singular vector) that capture the dynamics of the brain network in non-ictal and ictal states.

To construct models describing the evolution of multivariate statistics, we note that the network-based statistics evolve over time because of subclinical changes of brain activity that affect iEEG data in time and frequency. To estimate these changes, we can model the evolution of each SVD-based statistic through a hidden Markov model (HMM). The HMMs can be estimated from data for each patient and will characterize the neural dynamics in non-ictal and ictal states, and the probability distribution of the actual transition time (T) from any non-ictal to ictal state.

For some applications, for each patient, iEEG recordings can be used to (i) construct a time sequence of connectivity matrices, {A(k)}, (ii) compute the SVD for each matrix, and (iii) track the leading singular value, {σ₁(k)}, and the corresponding leading singular vector, {u₁(k)}, which are used for OSD. These statistics are defined below and may significantly modulate during seizure.

Recent studies have introduced schemes that simultaneously analyze all the available recording channels. In these schemes, each channel is treated as a node in a graph, and any two nodes are considered connected (i.e., an edge exists between them) if the activity at these sites are dependent. The connectivity (topology) of the graph can then be described by a matrix, which is referred to as the “connectivity” or “adjacency” matrix. Statistics computed from this matrix can show if the topology changes significantly from non-ictal to ictal states and significant changes in these statistics can be used to detect a seizure's onset.

The connectivity matrix, A, can be computed in several ways. In order to capture linear dependencies between all of the recording sites, we can compute connectivity as the cross-power in different frequency bands (theta, alpha etc.) between all available iEEG channels. That is, for each pair of channels (i, j) the corresponding element of the connectivity matrix in the frequency band Bᵣ is

$${a}_{ij , {B}_{r}} {=}^{Δ} {\int }_{{B}_{r}} {P}_{ij} ( f ) d f \qquad ( 1 )$$

where Pᵢⱼ(f) is the cross-power spectral density of channels i and j at frequency f. The frequency bands will include: B₁=[4,7] Hz (theta band), B₂=[8,13] Hz (alpha band), B₃=[13,35] Hz (beta band), and the final non-square connectivity matrix will be (for these bands)

$$A {=}^{Δ} [ \begin{matrix}{A}^{{B}_{1}} & {A}^{{B}_{2}} & {A}^{{B}_{3}}\end{matrix} ] . \qquad ( 2 )$$

One can compute A over a sliding window, which will result in a sequence of matrices {A(k)}, one per window. Based on data provided later in this section, one can initially use 5 s-long windows with 1 s slide, which will result in a connectivity matrix A(k) at each time second k.

### Singular Value Decomposition (SVD)

Measures can be computed based on the connectivity matrix (2) to generate some multivariate statistics that significantly modulate in the ictal state. Specifically, it has been suggested that the brain enters a more organized, lower complexity state prior to a seizure. As the brain becomes more organized and nodes become more connected, the rank (that is, the number of linearly independent rows or columns) of the connectivity matrix drops. The SVD of a matrix highlights the rank of a matrix and, in addition to that, it generates a weighted set of vectors that span the range space and null space of the matrix. Therefore, we can use SVD to measure the time-varying complexity of the brain by tracking the rank and its associated subspaces as a means to characterize non-ictal and ictal states. The SVD of the m×n connectivity matrix A is defined as

$$A = U S {V}^{*} = [ \begin{matrix}{u}_{1} & {u}_{2} & … & {u}_{m}\end{matrix} ] [ \begin{matrix}{σ}_{1} & 0 & … & 0 \\ 0 & ⋱ & … & 0 \\ ⋮ & ⋮ & {σ}_{q} & ⋮ \\ 0 & 0 & … & 0\end{matrix} ] [ \begin{matrix}{v}_{1}^{*} \\ {v}_{2}^{*} \\ ⋮ \\ {v}_{n}^{*}\end{matrix} ] \qquad ( 3 )$$

where U is an m×m unitary (UU*=I) matrix whose columns, uᵢ, are the eigenvectors of the matrix AA*, V is an n×n unitary matrix whose columns, vᵢ, are the eigenvectors of the matrix A*A, and * denotes the complex conjugate transpose operator. S is an m×n matrix whose first q diagonal entries σ₁≧σ₂≧ . . . ≧σ_{q} are the nonzero singular values of A, with q being the rank of A. The first q columns of U span the column space of A and the first q rows of V span the row space of A. When m=n and A is square symmetric (A=A*), SVD reduces to the conventional eigenvalue decomposition, where the singular values are the square of the eigenvalues of A, U=V⁻¹, and the columns of U and V are the eigenvectors of A.

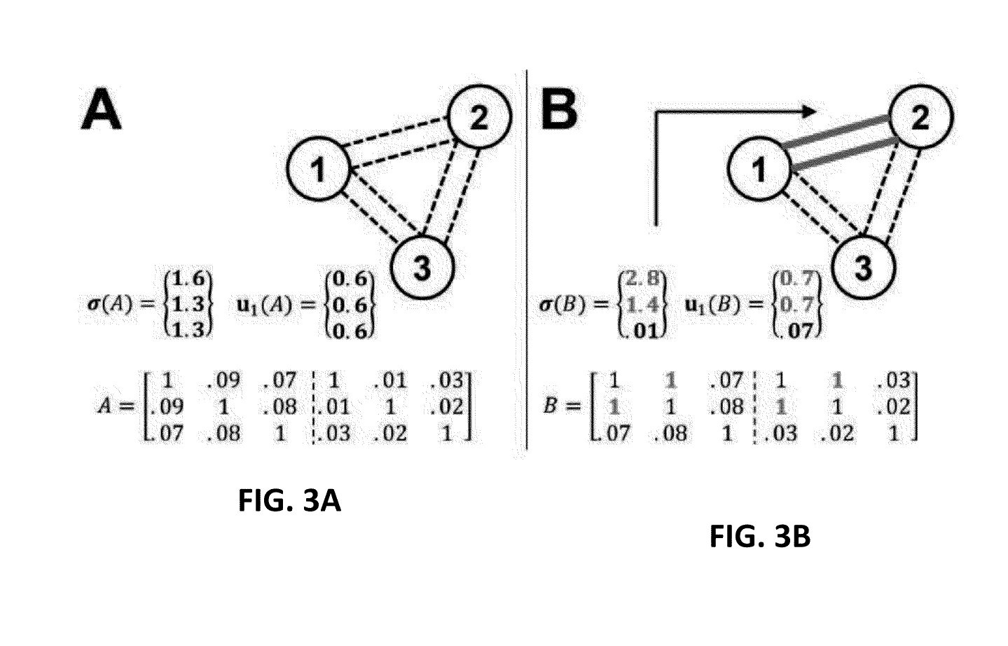

An example is shown in FIG. 3. Here, two 3-node graphs are analyzed. In FIG. 3A, all nodes have similar weak connections (strength <1) under two measures (e.g., cross-power in two frequency bands). The SVD of the corresponding non square connectivity matrix, A, reveals that the matrix of this graph has full rank (three comparable singular values in σ(A)). More physically, full rank here indicates the activity in the 3 nodes spans a 3 dimensional space, or has 3 degrees of freedom. If the strength between nodes 1 and 2 increases (FIG. 3B), one of the singular values of the corresponding connectivity matrix, B, becomes small in comparison to the other two, indicating that the rank of the matrix has approximately dropped to 2. This means that with the addition of one strong connection, the activity on the graph collapses to two dimensions and has becomes more “ordered”. The singular vectors of graphs in FIG. 3 are given and indicate that the dominant direction of the vectors has also rotated in FIG. 3B.

The time dependent structure of the first singular vector direction of the connectivity matrix can be used as way to detect seizure onsets.

### Example Results

Data: Four subjects with intractable epilepsy were surgically implanted with subdural grid and strip electrodes (26-75 channels, Ad-Tech® Medical Instrument Corporation, Racine, Wis.) for approximately one week before surgical resection of the focal region and monitored by clinicians for seizures and inter-ictal epileptic activity. Electrodes are 4 mm diameter platinum contacts embedded in a silicone sheet with 2.3 mm exposed. Data were digitized and stored using an XLTEK® EMU128FS system (Natus Medical Incorporated, San Carlos, Calif.) with 250-500 Hz sampling frequency. Table I reports patient-specific information, number of electrodes included in this study, and electrode position, respectively. Up to three board-certified epileptologists marked the unequivocal electrographic onset (UEO) by consensus of each seizure and the period between onset and termination. UEOs were used as the “gold standard” for evaluating the performances of the detection algorithm. Grid-electrode iEEG recordings included in this study were made available with the written consent of the patients, in accordance with the protocol approved by the Institutional Review Boards at Brigham and Women's Hospital and Children's Hospital, Boston, Mass.

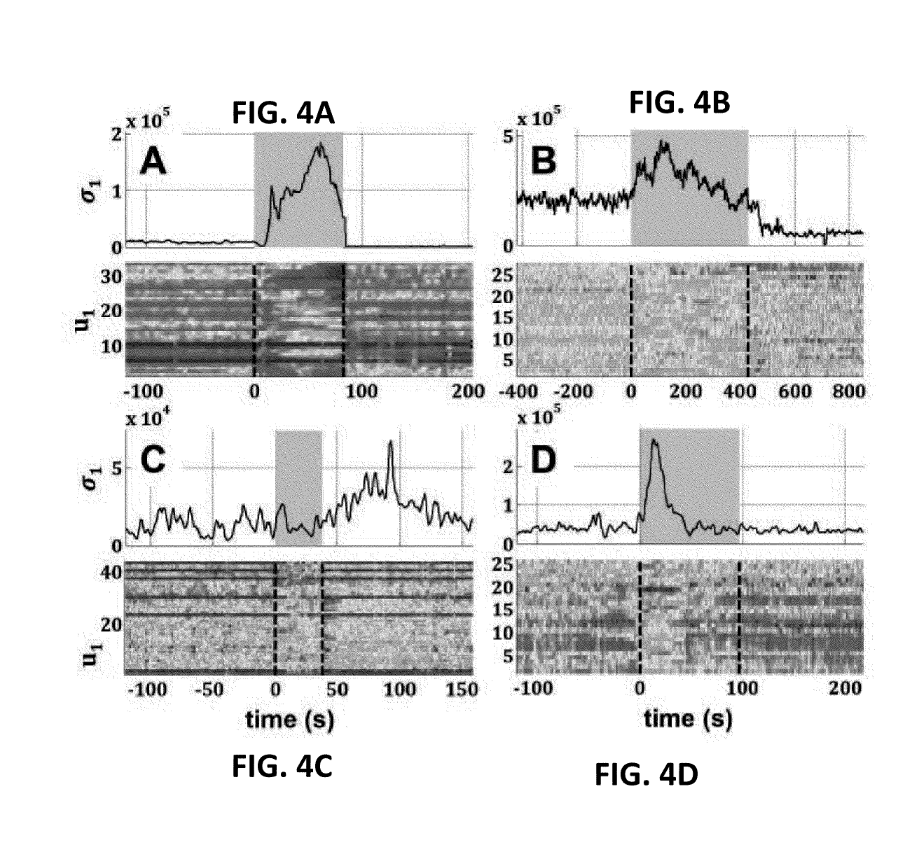

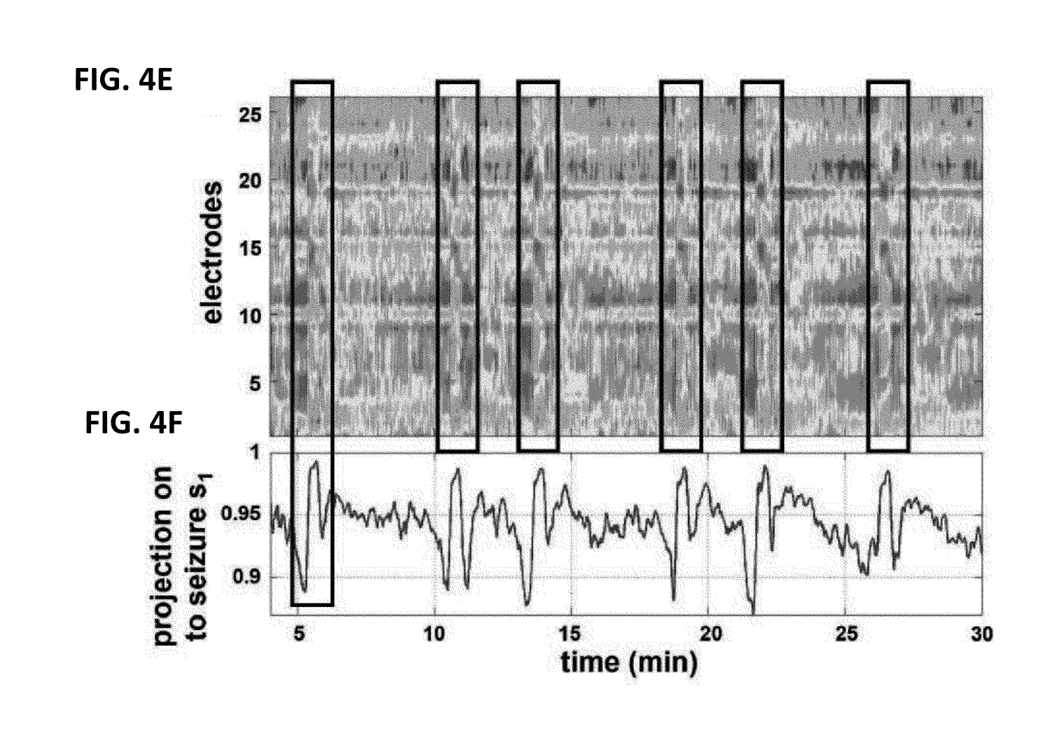

SVD Statistics: The connectivity matrix was estimated using the cross-power in a specific frequency band (1) for each patient over consecutive overlapping windows (5 s-long window, 4 s overlap). See Table I. We computed connectivity in one frequency band for simplicity to initially construct our QD framework. The corresponding maximum singular value σ₁ and first singular vector u₁ are plotted in FIG. 4 for consecutive windows covering an ictal period.

TABLE I
Experimental setup. F = frontal lobe; O = occipital
lobe; T = temporal lobe; CP = complex partial;
SP = simple partial; TC = tonic clonic. For each
patient, the frequency band was chosen by maximizing the
distance between ictal vs. non-ictal GLM parameters (training data only).

| Patient | Seizure | Type of | # iEEG | hours of | Frequency |
|---|---|---|---|---|---|
| ID | origin | seizures | channels | recordings | band for σ₁ |
| 1 | T | CP | 34 | 40 | 13-30 Hz |
| 2 | T | TC | 28 | 47 | 4-7 Hz |
| 3 | F | CP | 44 | 47 | 13-30 Hz |
| 4 | O | SP | 26 | 34 | 13-30 Hz |

The sequence of σ₁ has a consistent pattern across patients during the non-ictal, pre- and post-ictal states. The corresponding singular vector u₁ shows a leading direction before the seizure onset, which depends on both the patient and the location of the focal region. During a seizure, σ₁ rapidly increases compared to the non-ictal activity in the previous minutes, reaches a local maximum at approximately half of the ictal period (gray boxes, FIG. 4A-D), and then slowly decreases to smaller non-ictal values. The change in the dynamics of σ₁ is observed almost at the beginning of the hand-annotated seizure onset, while the return to the non-ictal condition is usually slower. Interestingly, after every seizure, σ₁ decreased below the average value achieved before the seizure and, then, increased to the pre-ictal values with a long drift (at least 2 hours, data not reported), which may be consistent with the definition of a post-ictal state.

The stereotypical dynamics of σ₁ was associated with a sudden change in the direction of the singular vector u₁. Furthermore, the new direction remained consistent across different seizures (FIG. 4E,F). Modulo a scaling factor, the dynamics of σ₁ and u₁ were similar in patients 1-2, and 4 (FIG. 4A,B,D), independently of the connectivity matrix being computed in different frequency bands. These dynamics were less clear in patient 3, where σ₁ showed slow oscillations independently of the seizure occurrence (FIG. 4C, top). However, at the seizure onset, the value of σ₁ first decreased, then rapidly rose to a local maximum, and finally drifted to baseline value, as did occur in the other patients.

The notion of connectivity (e.g., cross-power) and the particular statistic computed from the connectivity matrix (e.g., σ₁) may not perform well for all types of seizures or all types of epilepsy. If needed, one can use other connectivity measures (e.g., the mutual information) and/or other matrix measures (e.g., spectral clusters). Furthermore, there may be patients who have independent multifocal seizure onsets, where the presence of multifocal seizures may determine a connectivity matrix that changes periodically over time as these seizures ping-pong back and forth between foci. To identify these cases, the consistency of the seizure onset pattern will be measured across all seizures within a patient.

### Modeling the Evolution of the Multivariate Statistics

In one embodiment, we model the evolution of the maximum singular value statistic using an HMM. For any given patient, we assume that the maximum singular value computed at each second is generated by an HMM. In particular, at each stage k≧0, the brain is in one of m subclinical states, i.e., xₖ ∈ {0,1, . . . , m−1} which follows a Markov Chain, i.e.,

$$Pr ( {x}_{k + 1} = j \vert {x}_{k} , {x}_{k - 1} , … , {x}_{0} ) = Pr ( {x}_{k + 1} = j \vert {x}_{k} = i ) {=}^{Δ} {p}_{ij} \\ for all i , j ∈ \{ 0 , 1 , 2 , … , m - 1 \} \\ {\sum }_{j = 0}^{m - 1} {p}_{ij} = 1 \\ for all i ∈ \{ 0 , 1 , 2 , … , m - 1 \} \\ {\sum }_{j = 0}^{m - 1} {℘}_{j} = 1 \\ where \\ {℘}_{j} {=}^{Δ} Pr ( {x}_{0} = j ) , \qquad ( 4 )$$

j=0,1, . . . , m−1, is the probability of starting in state j. For a fixed state j, we assume that the observations

$${z}_{k} {=}^{Δ} {σ}_{1} ( k ) ,$$

k=0,1,2, . . . , are generated according to a known history-dependent probability law

$${q}_{j} ( z \vert {H}_{k} ) {=}^{Δ} Pr ( {z}_{k} = z \vert {x}_{k} = j , {H}_{k} ) \qquad ( 5 )$$

where the sequence of past observations is given in

$${H}_{k} {=}^{Δ} \{ {z}_{0} , {z}_{1} , … , {z}_{k - 1} \} .$$

Note mat the i dependency of zₖ on previous observations accounts for temporal dependencies that exist in neural data. The HMM is therefore uniquely defined by the triple { [symbol] , Σ, q}, with

$$𝒫 {=}^{Δ} [ \begin{matrix}{℘}_{0} & {℘}_{1} & … & {℘}_{m - 1}\end{matrix} ] , \\ {\sum }_{ij} {=}^{Δ} {p}_{ij} ,$$

i,j=0, . . . , m−1, and

$$q {=}^{Δ} [ \begin{matrix}{q}_{1} & {q}_{1} & … & {q}_{m - 1}\end{matrix} ] .$$

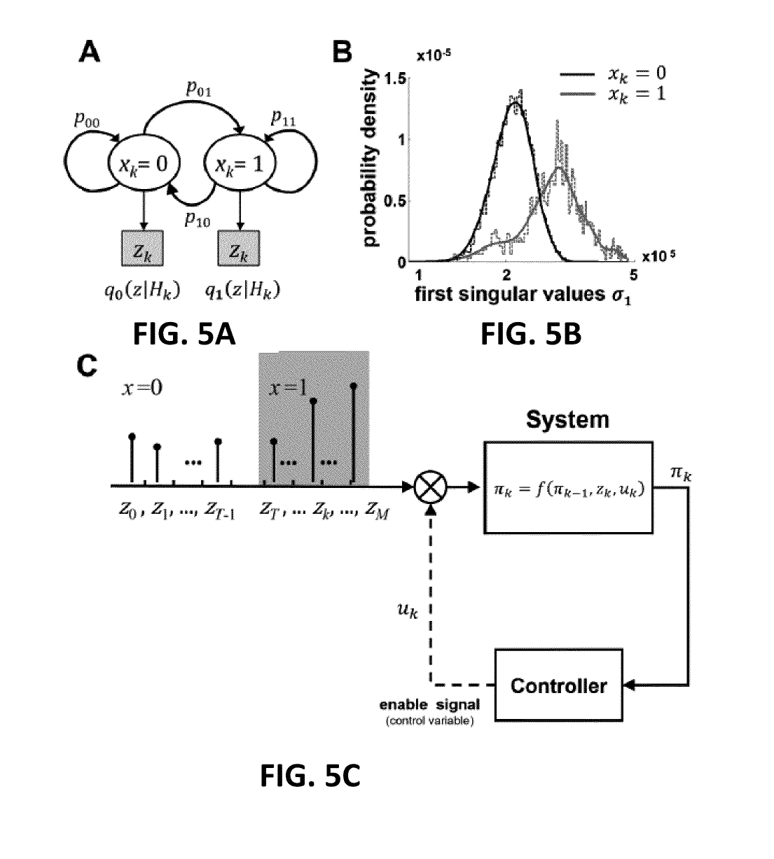

### See FIG. 5A.

For our QD framework, we may initially fit an m=2 state HMM on each patient, with state x=0 and x=1 denoting the non-ictal and ictal condition, respectively. The ictal state begins and ends with the unequivocal ictal onset and offset determined by clinicians. Early-ictal or pre-ictal conditions are subsumed in the non-ictal state as they may not exist in all patients. Since we will begin monitoring a patient in the non-ictal state 0, we set [symbol] =[1 0]. We will also initially assume that the state transition probability matrix is

$$\sum = [ \begin{matrix}1 - ρ & ρ \\ 0 & 1\end{matrix} ] \qquad ( 6 )$$

where ρ=p₀₁ will be estimated from training data via maximum likelihood estimation. The output probability law qₓ(z|Hₖ), x=0,1, will be estimated by combining generalized linear models (GLM) and maximum likelihood estimation. Training data includes at least 3 hours of non-ictal data well before seizure (min 3 hours, max 12 hours before seizure) and at least one seizure.

### Example Results

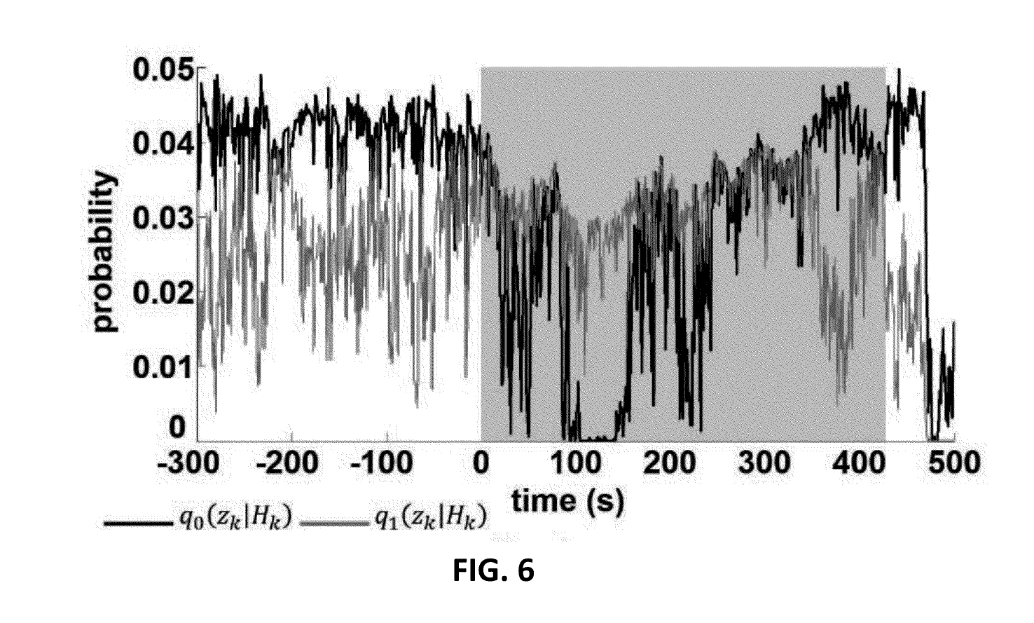

We applied the HMM modeling to data described in [0042]-[0046]. FIG. 5B and FIG. 6 show results for the HMM estimation. Although the mean value and the variance of σ₁ were different in the ictal versus non-ictal state, the sampling probability distribution functions overlap (FIG. 5B), which means that several of the same values of σ₁ were likely to be achieved both during the ictal and non-ictal states.

To better characterize the distribution of σ₁ in each state, we used a history-dependent model to describe the distribution of σ₁ (FIG. 6). At each stage k, this model modulates the probability of observing σ₁(k)in each state based on the values of σ₁ in the last 15 s. The functions q₀ and q₁ (i) varied the probability of any given observation σ₁ at each stage k depending on the past observations, and (ii) had opposite dynamics in ictal versus non-ictal state. For the computed sequence of σ₁ in each patient, q₁(·) was consistently larger than q₀(·) during ictal periods, but decreased during non-ictal periods (FIG. 6). In each patient, q₀(·) and q₁(·) were almost 0 right after every seizure, suggesting a post-ictal period characterized by a resetting of brain activity.

There may be reactivity of iEEG waveforms due to sleeping, moving, etc., which affects how the multivariate statistic evolves over time. To capture reactivity, it may needed to build an m-state HMM, where m>2, for each patient (e.g., m=3, with non-ictal sleep, non-ictal awake, and ictal state). The methodology described above is easily extended to such cases.

### Quickest Detection of Seizure Onsets

We will (i) implement the QD framework, (ii) test the QD-based strategy on clinical data, and (iii) compare QD to a variety of existing OSD algorithms. We begin by deriving the QD framework and then describe other OSD algorithms that we will also implement for comparison. We then discuss the results of the examples according to an embodiment of the current invention.

Because the state of an HMM is hidden, a Bayesian information state variable

$${π}_{k} {=}^{Δ} Pr ( {x}_{k} = 1 \vert {z}_{k} , {H}_{k} )$$

can be introduced in order to estimate how likely the transition from the non-ictal to ictal state is at each stage k. Note that πₖ is the a posteriori probability of being in state 1 at stage k and depends on the observations up to and including stage k. The evolution equation of πₖ is recursive and given by

$${π}_{k + 1} = \frac{{L}_{k + 1} [ {π}_{k} + ( 1 - {π}_{k} ) {p}_{01} ]}{( 1 - {p}_{01} ) ( 1 - {π}_{k} ) + {L}_{k + 1} [ {π}_{k} + ( 1 - {π}_{k} ) {p}_{01} ]} {=}^{Δ} {Φ}_{k + 1} ( {π}_{k} , {z}_{k + 1} , {H}_{k + 1} ) \qquad ( 7 )$$

with initial condition π₀=P(x₀=1|z₀)=q₁(z₀) [symbol] /[q₀(z₀) [symbol] +q₁(z₀) [symbol] ] and

$${v}_{0} {=}^{Δ} 1 - {0}_{} .$$

In these formula, qₓ(z₀) is the probability of observing z₀ in state x ∈ {0,1} at time k=

$$0 , {L}_{k} {=}^{Δ} {q}_{1} ( {z}_{k} \vert {H}_{k} ) / {q}_{0} ( {z}_{k} \vert {H}_{k} )$$

is the likelihood ratio, and the Bayes' rule is applied. Note that the evolution equation (7) depends on the likelihood ratio, Lₖ , between q₁ and q₀. The dependency of q₁ and q₀ on the history Hₖ contributes to separating their dynamics, thus achieving a larger modulation of Lₖ in ictal versus non-ictal states. Consequently, this history-dependency makes πₖ more reliable as it achieves high values only around the actual seizure onset.

The quickest detection problem is an online decision problem, where at each stage k, we test the hypothesis

$${=}^{Δ} \{ a seizure onset has occurred \}$$

conditioned on the observations (Hₖ, zₖ). We introduce the decision variable uₖ ∈ {0,1}, where uₖ=0 (uₖ=1) denotes that the hypothesis [symbol] is rejected (accepted) at stage k. In this way:

$${π}_{k + 1} = {f}_{k + 1} ( {π}_{k} , {z}_{k + 1} , {H}_{k + 1} , {u}_{k} ) {=}^{Δ} \{ \begin{matrix}{Φ}_{k + 1} ( {π}_{k} , {z}_{k + 1} , {H}_{k + 1} ) & {u}_{k} = 0 \\ terminate \& restart & {u}_{k} = 1\end{matrix} \qquad ( 8 )$$

where the “terminate & restart” state implies that we restart the detection algorithm after a seizure is detected. With this setup, QD boils down to deciding when to switch from uₖ=0 to uₖ=1, thus claiming that a seizure has occurred. We will design a decision strategy that minimizes the following cost function, which weighs average detection delay and probability of a false positive:

$${𝒥}_{0} {=}^{Δ} {a}_{1} {E}_{T} \{ ϕ ( T - {T}_{S} ) \} Pr ( T > {T}_{S} ) + {a}_{2} {E}_{T} \{ {ϕ}^{2} ( {T}_{S} - T ) \} Pr ( T \leq {T}_{S} ) \qquad ( 9 )$$

where T and T_{S} are the actual and estimated electrographic onset of the seizure, respectively, and φ(ε) is a nonnegative non-decreasing function of

$$ε {=}^{Δ} T - {T}_{S}$$

(in case of early detection, i.e.,

$$T > {T}_{S} ) or ε {=}^{Δ} {T}_{S} - T$$

(in case of delayed detection, i.e., T_{S}≧T), and is 0 otherwise. E_{T}{·} is the expected value and a₁, a₂ are parameters to be chosen to trade-off early versus delayed detection. For example, a₁=1−γ and a₂=γ, where the parameter γ ∈ [0,1] allows the tradeoff of false positives and delayed detection. Note that T is unknown but its probability distribution is defined by the HMM transition probabilities, i.e., P(T=k)=(1−p₀₁)^{k-1}₀₁. Also, note that the absolute distance |T−T_{S}| is weighted differently in case of early versus delayed detection (linear versus quadratic value of φ) in order to penalize long delays, which would be unacceptable.

We then design the cost (9) as a function of the information state πₖ and decision variable uₖ. Then, the optimal decision deals with choosing the stage T_{S}>0 such that the policy (u₁=0, u₂=0, . . . , u_{T}_{_{S}}₋₁=0, u_{T}_{_{S}}=1) minimizes the overall cost (9) with respect to the variable uₖ given the evolution model (8). The optimal decision is formulated as an optimal feedback control problem where uₖ is the control variable (FIG. 5C). This formulation can be solved recursively via dynamic programming, and leads to the optimal quickest detection policy

$${T}_{QD} {=}^{Δ} {T}_{S}^{*} = min \{ 0 < k < M \vert {π}_{k} > {F}_{k} ( {π}_{k} , {z}_{k} , {H}_{k} ) \} \qquad ( 10 )$$

where Fₖ(πₖ, zₖ, Hₖ) is an adaptive threshold that depends on the current observation, history, and information state variable, and M is the limit time before which a decision must be taken (M can also be infinite, which means that no time constraint is posed). The threshold Fₖ(·) is computed recursively and decreases over time non-monotonically.

### Example Results

We applied the QD framework to the data described in [0042]-[0046]. For each patient, we compare the QD policy to a classical Bayesian estimator (BE), which is widely used in the field of change-point detection, and a heuristic threshold based detector (HT), where the threshold is chosen heuristically. The formulae for the estimated seizure onset with each of these predictors are:

$$BE \text{:} {T}_{BE} {=}^{Δ} min \{ k > 0 \vert {π}_{k} > 0.5 \} and$$

$$HT \text{:} {T}_{HT} {=}^{Δ} min \{ k > 0 \vert {z}_{k} > \overline{h} \} ,$$

where the threshold h̅ is fixed heuristically. For each detection policy, we measure the delay between each estimated seizure onset time and the unequivocal electrographic onset, which will be annotated by the epileptologists. We can also evaluate the number of true positives (TP), false positives (FP), and false negatives (FN) per patient, where each decision can be classified as TP or FP if an unequivocal onset occurs within a window W from the detection time or not. W was initially be set to 20 s. A seizure onset not detected is classified as FN. Finally, given TP, FP, and FN, two measures can be evaluated for each patient: (i) the false positive rate (FPR), which is the number of FP/hour, and the “sensitivity” (S), which is the ratio between TP and TP+FN. For each of these measures, we determine whether the results achieved with the QD policy and any other method are significantly different from the chance-level detection (i.e., random generator of warnings) and whether FPR and S with QD are significantly lower than with the other methods (t-test). We can finally evaluate the sensitivity to changes of W as well as QD

TABLE II
Performance Analysis.

| | | | QD | | | | | BE | | | | | HT | | | | |
|---|---|---|---|---|---|---|---|---|---|---|---|---|---|---|---|---|---|
| | | Sei- | Specificity | | | Sensitivity | | Specificity | | | Sensitivity | | Specificity | | | Sensitivity | |
| | Patient | zures | FP | FPR (FP/h) | FN | TP | ΔT (s) | FP | FPR (FP/h) | FN | TP | ΔT (s) | FP | FPR (FP/h) | FN | TP | ΔT (s) |
| Training | 1 | 1 | 0 | 0 | 0 | 1 | 18 | 0 | 0 | 0 | 1 | 16 | 8 | 2.67 | 0 | 1 | 14 |
| | 2 | 1 | 0 | 0 | 0 | 1 | 19 | 0 | 0 | 0 | 1 | 16 | 8 | 2.67 | 0 | 1 | 20 |
| | 3 | 1 | 3 | 1 | 0 | 1 | 10 | 3 | 1 | 0 | 1 | 7 | 8 | 2.67 | 1 | 0 | n.a. |
| | 4 | 2 | 0 | 0 | 0 | 2 | 9 | 0 | 0 | 0 | 2 | 7 | 0 | 0 | 0 | 2 | 5 |
| Validation | 1 | 1 | 6 | 0.16 | 0 | 1 | 15 | 6 | 0.16 | 0 | 1 | 14 | 42 | 1.14 | 0 | 1 | 12 |
| | 2 | 2 | 7 | 0.16 | 0 | 2 | 31 | 11 | 0.25 | 0 | 2 | 33 | 13 | 0.30 | 0 | 2 | 14.5 |
| | 3 | 3 | 130 | 2.95 | 0 | 3 | 13.7 | 320 | 7.27 | 0 | 3 | 10.7 | 75 | 1.71 | 3 | 0 | n.a. |
| | 4 | 22 | 71 | 2.29 | 0 | 22 | 11.8 | 138 | 4.45 | 0 | 22 | 9.7 | 277 | 8.94 | 0 | 22 | 5.8 |

FP = false positive;
TP = true positive;
FN = false negative;
FPR = false positive rate;
n.a. = not available.
ΔT = |T_{S} − T|.
Note that absolute detection delays and anticipation “delays” are not shown.

performance for different trade-off gains γ in (9), where a₁=1−γ and a₂=γ.

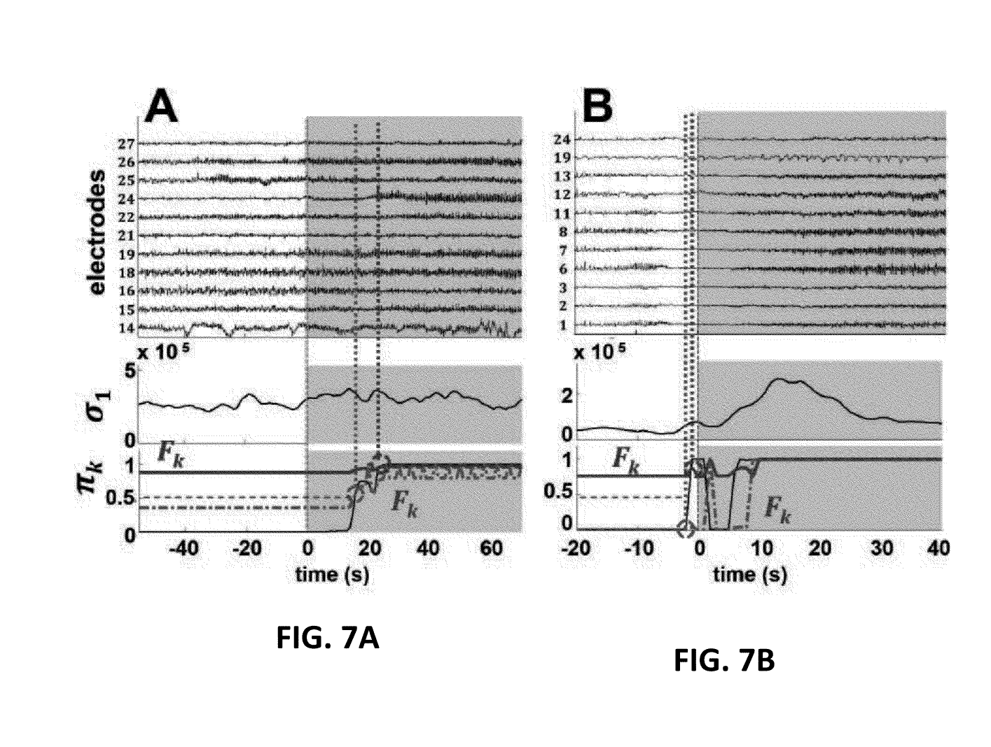

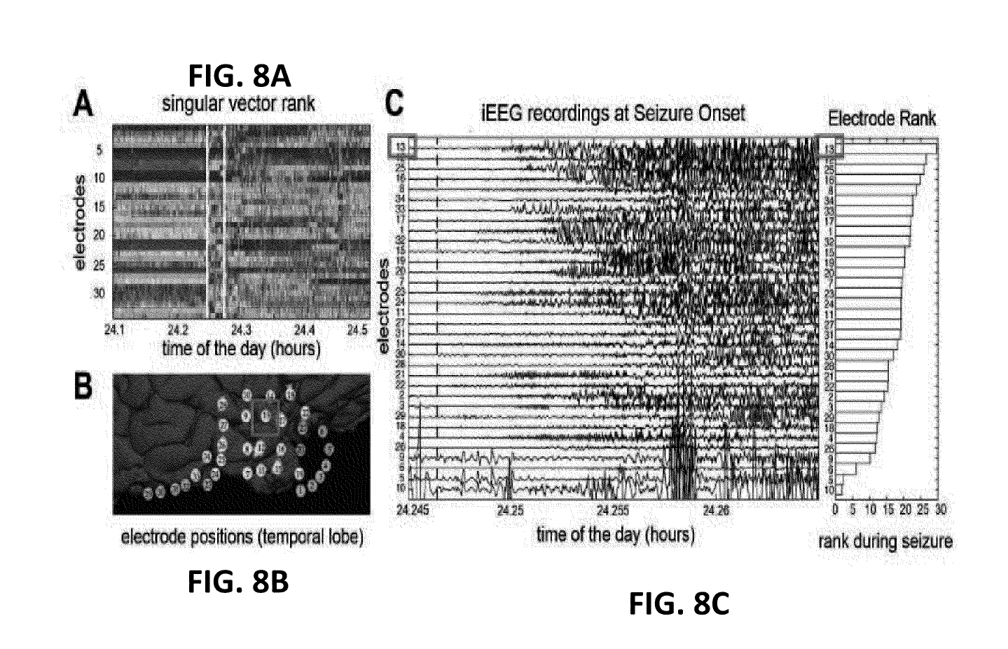

Table II and FIG. 7 report results for the QD policy vs. the BE and HT detectors. QD achieved significantly fewer false positives than the Bayesian and threshold-based detector (min. 36% less; max. 85% less; mean: 58% less), while achieving 100% sensitivity in every patient. QD delays were comparable and in some cases slightly larger than delays of other detectors. However, FIG. 7A shows that for patient 2, by increasing the penalty for detection delay in (9) (i.e., increasing γ), QD reduced delays to values achieved by BE (16 s, green lines), while maintaining a lower number of FPs (7 vs. 11). For patient 4, by decreasing γ, QD achieved higher robustness to early modulations in the probability πₖ, due to abrupt spikes in the sequence of σ₁, and QD decreased the number of FPs and detect a seizure with less anticipation (FIG. 7B).

QD results in significantly fewer FPs than other OSD methods, however, the detection delays, are comparable to those produced by other OSD methods. One can also explore penalizing other functions of the detection delay |T_{S}−T| in the cost function (9) to reduce these delays according to other embodiments of the current invention. For example, one can allow this penalty to grow exponentially (e^{|T}^{_{S}}^{-T|}) and as long as the function is a non-decreasing function of the delay, the QD method will hold.

### Multi-Component Multivariate Statistics

In another embodiment, the multivariate statistics computed off the connectivity matrix is a vector of two or more components, i.e., the observation zₖ in (5) at each stage k is a vector of two or more components instead of a scalar. For example, zₖ can characterize the distribution of the whole set of singular values of the connectivity matrix A in (2) at every stage k. To do so, it can be

$${z}_{k} {=}^{Δ} {[ {ξ}_{k} , {v}_{k} ]}^{T} ,$$

where ξₖ and υₖ are the median and standard deviation of the distribution of the singular values at stage k, respectively. In this embodiment, the distribution function qⱼ(z|Hₖ), j=0,1, . . . , m−1, in (5) will be a vector function with as many components zₖ, where each component will be separately modeled.

In another embodiment, the observation zₖ in (5) at each stage k is a vector and describes the “singular vector centrality” (SVC) of the nodes of the brain network, whereas each node is given by one of the recording iEEG channels. SVC is analogous to eigenvector centrality (EVC) for square symmetric connectivity matrices and provides a measure of the importance of a given node in the network, i.e., it says how densely connected that node is to other nodes. EVC measures the importance of a node in a network. Further, EVC assigns scores to all nodes in the network based on the principle that a connection to a high scoring node contributes more to the node in question than equal connections to low scoring nodes. Differently from the EVC, the SVC of a node is proportional to the sum of the SVCs of its neighbors (nodes it is connected to) across multiple frequency bands, i.e., a node is important either if (i) it is connected to a few nodes that are themselves very important in some or all bands, or if (ii) it is connected to a very large number of not-so-important nodes in some or all bands.

The SVC notion is mathematically formalized as follows: denoted with s(i) the SVC of node i, it is s(i) ∝ Σ_{j∈n(i)}āᵢⱼs(j), where n(i) is the set of nodes connected to node i and āᵢⱼ is the (i,j)-th element of AA*. In matrix form s ∝ (AA*)s and this property holds when s is an eigenvector of AA*. It can be shown that the eigenvector corresponding to the largest eigenvalue of AA* is the steady state SVC and that, for non-square matrices, it is the left singular vector, i.e., s=u₁, see (3). Therefore, the components in u₁ with larger magnitude during seizure identify the nodes with stronger dependencies and these nodes could mark the initial stages of a seizure, thus allowing an early detection of the seizure onset.

In another embodiment, the observation zₖ in (5) at each stage k is a ranked version of the SVC vector u₁(k) at stage k. In particular, denoted with N the number of nodes in the network (i.e., the number of recording iEEG electrodes), u₁(k) has N components and is converted to a ranked vector containing values 1 to N, where 1 corresponds to the component of u₁(k) with the largest SVC and N is placed in the component of u₁(k) that has the smallest SVC value. In this way, a time series of ranks for each electrode {yₖ(i)}_{k=1,2, . . .} , i=1, . . . , N, where yₖ(i) ∈ {1,2, . . . , N} is the rank of electrode i at time k. First, {yₖ(i)}_{k=1,2, . . .} is normalized to emphasize the temporal evolution of the rank:

$${\overline{y}}_{k} ( i ) {=}^{Δ} ( {y}_{k} ( i ) - {\hat{μ}}_{i} ) / ‖ {y}_{k} ( i ) - {\hat{μ}}_{i} ‖ \qquad ( 11 )$$

where \hat{μ}ᵢ is the moving time-average of the rank signal and ∥·∥ is the 2-norm. Second, the series y̅ₖ(i), i=1, . . . , N are used to compute the cross-correlation matrix C*, whose the generic (i,j)-th element is

$${C}_{ij}^{*} {=}^{Δ} {max}_{k} {\sum }_{h = 1}^{N} {\overline{y}}_{k - h} ( i ) {\overline{y}}_{h} ( j ) , \qquad ( 12 )$$

i.e., C*ᵢⱼ is the peak value of the correlation between the time series of electrodes i and j. Third, the electrodes are clustered based on the matrix C*. In particular, the rows and columns are permutated to generate a new matrix that is approximately block-diagonal, and electrodes within each block are clusterized into Q clusters via the Kernighan-Lin (KL) algorithm. Note that the number Q comes from the application of the KL algorithm. Finally, for each cluster cl_{q}, q=1,2, . . . , Q we define the mean rank

$${r}_{q} ( k ) {=}^{Δ} {\sum }_{h ∈ {cl}_{q}} {\overline{y}}_{k} ( h ) / {n}_{q} ,$$

where n_{q} is the number of elements in cl_{q}. In this way, signal r_{q}(k) gives the mean pattern for all the electrodes in cl_{q}.

The examples reported above are included merely as an illustration of the present method and are not intended to be considered limiting. These examples are some of many possible applications of the methods described above. Any other suitable application of the above described methods known to or conceivable by one of skill in the art could also be created and used.

The notion of connectivity used thus far (i.e., multi-band cross-power) and the particular statistics computed from the connectivity matrix (i.e., σ₁, u₁) may not perform well for all types of seizure or all types of epilepsy. In this case, for example, alternative connectivity matrices based on spectral coherence can be used. If necessary, other connectivity matrices known to or conceivable by one of skill in the art such as coherence, mutual information, phase correlation and other multivariate statistics like spectral clusters can also be explored.

It should be noted that a non-transitory computer readable medium can be programmed to execute the above described vector-based method for detecting the seizure onset. The non-transitory computer readable medium can be loaded onto a computing device or server. The computing device or server can also be in networked communication with a source of data for analysis by the program loaded onto the non-transitory computer readable medium. The computing device can take any suitable form known to one of skill in the art such as a personal computer, tablet computing device, smartphone, processor, server, etc. The non-transitory computer readable medium can either be loaded directly onto a hard drive of the computing device, can be on a separate hard disk or CD-ROM, can be on the server described above, another independent server, a network, or any other suitable configuration known to or conceivable by one of skill in the art.

A non-transitory computer readable medium is defined as any article of manufacture that contains data that can be read by a computer. Such computer readable media includes but is not limited to magnetic media, such as a floppy disk, a flexible disk, a hard disk, reel-to-reel tape, cartridge tape, cassette tape or cards; optical media such as CD-ROM and writeable compact disc; magneto-optical media in disc, tape or card form; and paper media, such as punched cards and paper tape. The non-transitory computer readable medium contains code such that the method described herein can be executed.

The embodiments discussed in this specification are intended to explain concepts of the invention. However, the invention is not intended to be limited to the specific terminology selected and the particular examples described. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A multichannel seizure detection system, for detecting an onset time of seizures in a subject comprising:
a signal interface adapted to receive multichannel electrical signals from said subject's brain;
a data processor configured to receive said multichannel electrical signals and detect an incoming seizure,
wherein the onset time of the seizure is determined using a singular vector centrality (SVC) of each one of the channels comprising the multichannel electrical signals.

2. The system of claim 1 further comprising the SVC of a channel being proportional to the sum of the SVCs of its neighbors.

3. The system of claim 2 further comprising the sum of the SVCs of the neighbors of a channel being taken across multiple frequency bands.

4. The system of claim 2 wherein a neighbor of a channel is another channel to which said channel is connected.

5. The system of claim 1 further comprising an alert being made when a channel is connected to a predetermined number of channels that are themselves categorized as very important in some or all bands.

6. The system of claim 1 further comprising an alert being made when a channel is connected to a predetermined large number of nodes categorized as not-so-important in some or all bands.

7. The system of claim 1 further comprising the SVC being mathematically formalized as a vector of components s(i), where s(i) is the SVC of node i and s(i) ∝ Σ_{j∈n(i)}āᵢⱼs(j), where n(i) is the set of nodes connected to node i and āᵢⱼ is the (i,j)-th element of AA*.

8. The system of claim 7 wherein an eigenvector corresponding to the largest eigenvalue of AA* defines the steady state SVC vector and, for non-square matrices, this is the left singular vector

9. The system of claim 8 wherein the channels in u₁ with larger magnitude during a seizure correspond to the nodes with stronger dependencies across multiple measures, which may indicate a location where the seizure starts first.

10. The system of claim 9 wherein the time-dependent structure of a leading singular vector and a leading singular value of a connectivity matrix are used to detect the onset of a seizure.

11. The system of claim 1 further comprising a N-dimensional SVC vector u₁(k) evaluated at each second k (N is the number of electrodes) being converted to a ranked vector containing values 1 to N, where 1 corresponds to the component of u₁(k) with the largest SVC and N is placed in the component of u₁(k) that has the smallest SVC value.

12. The system of claim 11 further comprising a time series of ranks being generated for each electrode {yₖ(i)}_{k=1,2, . . . ,} i−1, . . . , N , where yₖ(i) ∈ {1,2, . . . , N} is the rank of electrode i at time k.

13. A method for detecting seizures in a subject comprising:
obtaining multichannel electrical signals from said subject's brain;
transmitting the multichannel electrical signals from said subject's brain to a computing device, wherein said computing device is loaded with a non-transitory computer readable medium is programmed for:
detecting an onset time of a seizure,
determining the onset time of a seizure by using a singular vector centrality (SVC) of each one of channels comprising the multichannel electrical signals.

14. The method of claim 13 further comprising calculating the SVC of a channel such that it is proportional to the sum of the SVCs of its neighbors.

15. The method of claim 14 further comprising taking the sum of the SVCs of the neighbors of a channel across multiple frequency bands.

16. The method of claim 14 further comprising defining a neighbor of a channel as another channel to which said channel is connected.

17. The method of claim 13 further comprising making an alert when a channel is connected to a predetermined number of channels that are themselves categorized as very important in some or all bands.

18. The method of claim 13 further comprising making an alert when a channel is connected to a predetermined large number of nodes categorized as not-so-important in some or all bands.

19. The method of claim 13 further comprising formulating the SVC as s(i) ∝ Σ_{j∈n(i)}āᵢⱼs(j), where s(i) is the SVC of node i, n(i) is the set of nodes connected to node i, and āᵢⱼ is the (i,j)-th element of AA*.

20. The method of claim 19 further comprising using an eigenvector corresponding to a largest eigenvalue of AA* to define the steady state SVC, whereas, for non-square matrices, this eigenvector is the left singular vector u₁.

21. The method of claim 20 further comprising using the channels in u₁ with larger magnitude during a seizure to correspond to the nodes with stronger dependencies across multiple measures.

22. The method of claim 21 further comprising using the time-dependent structure of a leading singular vector and a leading singular value of a connectivity matrix to detect a seizure onset.

23. The method of claim 22 further comprising converting an N-dimensional SVC vector u₁(k) evaluated at each second k, with N being the number of electrodes, to a ranked vector containing values 1 to N, where 1 corresponds to the component of u₁(k) with the largest SVC value and N is placed in the component of u₁(k) that has the smallest SVC value.

24. The method of claim 22 further comprising generating a time series of ranks for each electrode {yₖ(i)}_{k=1,2, . . .} , i=1, . . . , N, where yₖ(i) ∈ {1,2, . . . , N} is the rank of electrode i at time k.
